(19) United States
(12) Patent Application Publication
(10) Pub. No.: US 2011/0118817 A1
(43) Pub. Date: May 19, 2011

(54) STENT DELIVERY SYSTEM

(75) Inventors: RICHARD C. GUNDERSON, MAPLE GROVE, MN (US); KATHY PRINDLE, ROBBINSDALE, MN (US); JASON LENZ, MAPLEWOOD, MN (US)

(73) Assignee: BOSTON SCIENTIFIC SCIMED, INC., MAPLE GROVE, MN (US)

(21) Appl. No.: 12/620,436

(22) Filed: Nov. 17, 2009

(51) Int. Cl. A61F 2/06

(52) U.S. Cl. 623/1.12

(57) ABSTRACT

Stent delivery systems, including self-expanding stent delivery systems, and methods for making and using the same. A self-expanding stent delivery system may include an inner member. An outer sheath may be slidably disposed about the inner member. A self-expanding stent may be disposed between the inner member and the outer sheath. A rolling membrane may be attached to the outer sheath and to the inner member. A lubricious component may be disposed adjacent the outer sheath for reducing friction between the outer sheath and the membrane.

### TECHNICAL FIELD

The present invention pertains to medical devices and methods for making and using medical devices. More particularly, the present invention pertains to stent delivery systems.

### BACKGROUND

A wide variety of intracorporeal medical devices have been developed for medical use, for example, intravascular use. Some of these devices include stent delivery systems. These devices are manufactured by any one of a variety of different manufacturing methods and may be used according to any one of a variety of methods. Of the known stent delivery devices and methods for making and using the same, each has certain advantages and disadvantages. There is an ongoing need to provide alternative stent delivery devices as well as alternative methods for making and using stent delivery devices.

### BRIEF SUMMARY

The invention provides design, material, manufacturing method, and use alternatives for stent delivery systems including self-expanding stent delivery systems. An example self-expanding stent delivery system may include an inner member. An outer sheath may be slidably disposed about the inner member. A self-expanding stent may be disposed between the inner member and the outer sheath. A rolling membrane may be attached to the outer sheath and to the inner member. A lubricious component may be disposed adjacent the outer sheath for reducing friction between the outer sheath and the membrane.

Another example self-expanding stent delivery system may include an inner member. An outer sheath may be slidably disposed about the inner member. A self-expanding stent may be disposed between the inner member and the outer sheath. A rolling membrane may be attached to the outer sheath and to the inner member. The rolling membrane may include a reinforcing member that may be configured to provide the rolling membrane with increased column strength such that the reinforcing member reduces buckling of the rolling membrane when the rolling membrane is rolled back upon itself.

Another example self-expanding stent delivery system may include an inner member. A sheath may be slidably disposed about the inner member. A self-expanding stent may be disposed between the inner member and the sheath. A rolling membrane may be attached to the sheath and to the inner member. The rolling membrane may include a radiopaque marker.

Another example self-expanding stent delivery system may include an inner member. An outer sheath may be slidably disposed about the inner member. A self-expanding stent may be disposed between the inner member and the outer sheath. A rolling membrane may be attached to the outer sheath and to the inner member. The rolling membrane may include a plurality of layers including a lubricious layer.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a partial cross-sectional side view of an example stent delivery system;

FIG. 2 is a partial cross-sectional side view of the example stent delivery system shown in FIG. 1 with an outer sheath that is partially retracted to being deployment of a stent;

FIG. 3 is a partial cross-sectional side view of another example stent delivery system;

FIG. 4 is a cross-sectional view of a portion of an example membrane;

FIG. 5 is a cross-sectional view of a portion of another example membrane;

FIG. 6 is a cross-sectional view of a portion of another example membrane;

FIG. 7 is a cross-sectional view of a portion of another example membrane;

FIG. 8 is a partial cross-sectional side view of another example stent delivery system;

FIG. 9 is a partial cross-sectional side view of the example stent delivery system shown in FIG. 8 with an outer sheath that is partially retracted to begin deployment of a stent;

FIG. 10 is a partial cross-sectional side view of a portion of the example stent delivery system shown in FIGS. 8-9 with an outer sheath that is refracted; and

FIG. 11 is a cross-sectional view of a portion of another example membrane.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

The ability to accurately deploy a stent within the vasculature may be an important feature considered when designing stent delivery systems. Some self-expanding stent delivery systems utilize a rolling membrane to improve deployment accuracy. In general, a rolling membrane is a structure that covers the stent during delivery and then “rolls” proximally to uncover the stent. The rolling motion of the membrane may help to reduce the likelihood that longitudinal shifting the stent may occur during deployment. Rolling membranes reduce longitudinal displacement of the stent that may occur when an outer sheath is proximally retracted to expose and deploy the stent in the vasculature.

Because stents (e.g., self-expanding stents) may have a tendency to exert an outward force on the membrane and/or the outer sheath, a potential exists that frictional forces may be created between the sheath and the membrane. These forces could result in the longitudinal displacement of the stent upon retraction of the outer sheath. In order to minimize the potential of these frictional forces being present, a clinician may inject a pressurized fluid into the lumen of the outer sheath so that the fluid is disposed between the membrane and the sheath. The pressurized fluid, which may include a lubricious or otherwise friction-reducing material (e.g., a lubricious hydrogel, saline, etc.), may be effective in reducing the potential of frictional forces influencing the position of the stent and, thus, may improve the accuracy of stent deployment.

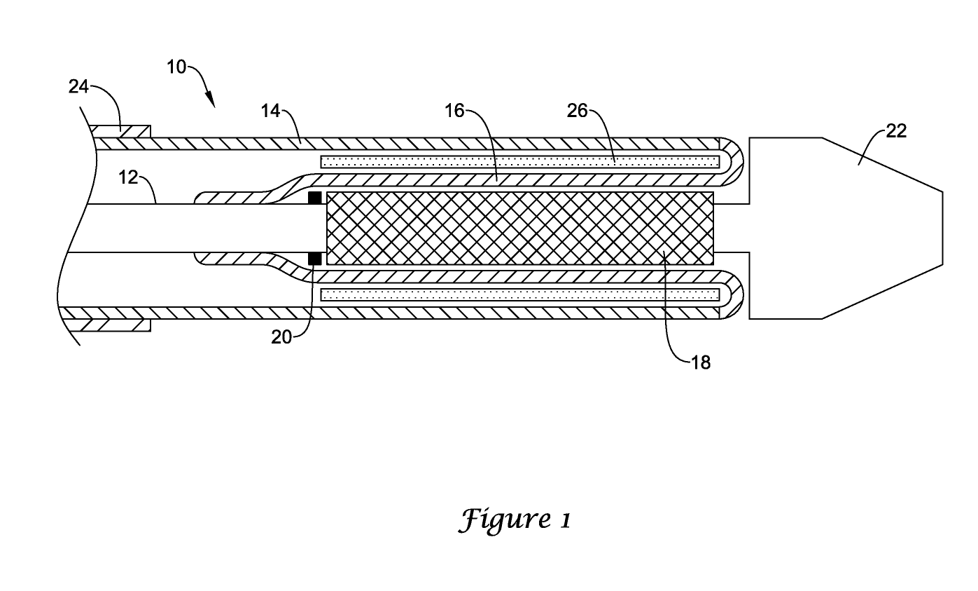

FIG. 1 illustrates an example stent delivery system 10 that is designed to have improved stent deployment accuracy. System 10 may include an inner member 12 and an outer sheath 14. A guidewire lumen (not shown) may be formed in inner member 12 so that system 10 may be advanced through the vasculature over a guidewire. A rolling membrane 16 may be attached to outer sheath 14 and inner member 12. For example, membrane 16 may attach to the outer surface of inner member 12 and to end surface (e.g., an abutting or butt-type joint) of sheath 14. A stent 18 may be disposed about inner member 12. In at least some embodiments, stent 18 is a self-expanding stent. Self-expanding stents are configured to shift from a radially compressed state to an expanded or deployed state suitable for expanding an intravascular lesion. A stent bumper 20 may be disposed on inner member 12, for example, adjacent the proximal end of stent 18. Bumper 20 may be formed of or otherwise include a radiopaque material and it may be configured to reduce proximal shifting of stent 18 that might otherwise occur during deployment of stent 18. An atraumatic tip 22 may be attached to or otherwise formed at the distal end of inner member 12. Some of the components of system 10 may be similar to those in U.S. Patent Application Pub. No. US 2006/0030923, the entire disclosure of which is herein incorporated by reference.

An outer member 24 may be disposed on a portion of sheath 14. Outer member 24 may be similar in form and function to similar structures disclosed in U.S. Patent Application Pub. No. US 2007/0208350, the entire disclosure of which is herein incorporated by reference. Outer member 24 may be longitudinally fixed relative to inner member 12 and may further aid in the reduction of friction between sheath 14 and membrane 16. System 10, as well as numerous other contemplated stent delivery systems, may lack outer member 24.

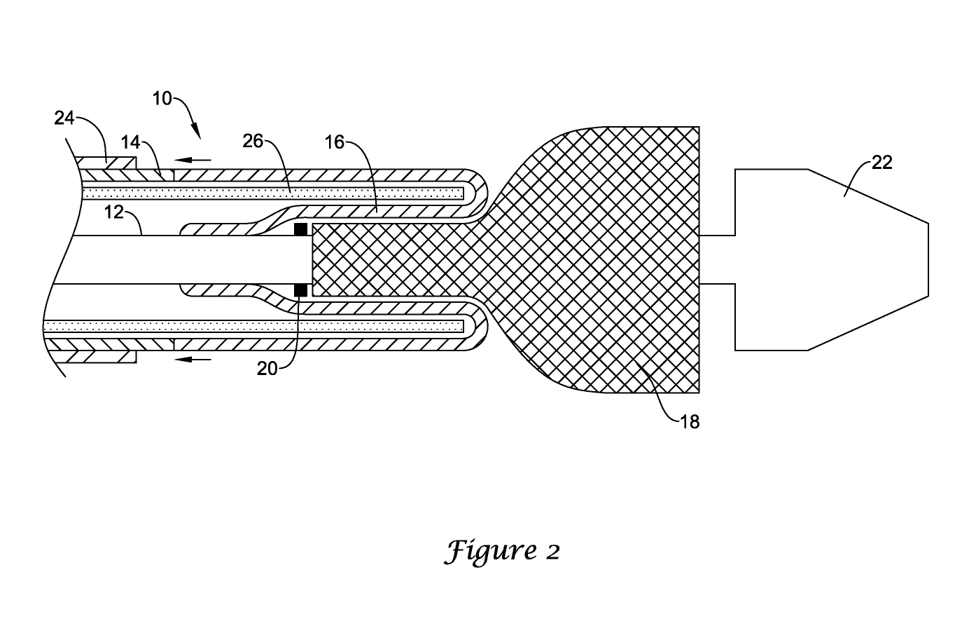

System 10 may also include a lubricious component 26. In general, lubricious component 26 is a structural feature (e.g., a member, component, part, element, feature, etc.) that is configured to reduce frictional forces between sheath 14 and membrane 16 that may be present when sheath 14 is retracted to deploy stent 18, for example as shown in FIG. 2. Thus, lubricious component 26 may help to improve the deployment accuracy of system 10 and allow stent 18 to be placed in the desired location within the vasculature with improved precision.

As the name suggest, lubricious component 26 may be formed or otherwise include a lubricious material. Because of this, lubricious component 26 may help reduce frictional forces between sheath 14 and membrane 16 and allow sheath 14 to be smoothly retracted while minimizing the possibility that stent 18 is displaced. Lubricious component 26 may vary in form. In at least some embodiments, lubricious component 26 is a tube (e.g., a “floating” tube) disposed between sheath 14 and membrane 16. By virtue of being a floating tube, lubricious component 26 may be understood to be free of attachment to essentially any component of system 10. Accordingly, lubricious component 26 may be free to move when sheath 14 is retracted without being encumbered by any other part of system 10.

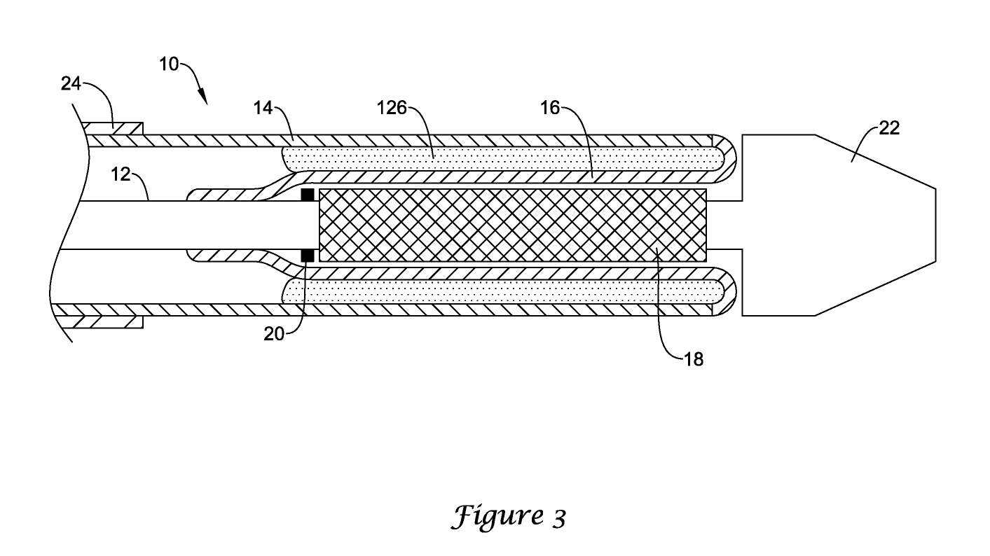

In alternative embodiments, for example as shown in FIG. 3, lubricious component 126 may take the form of a non-pressurized fluid or gel (e.g., a silicone lubricant such as SLIP-COAT® or the like) that is disposed between sheath 14 and membrane 16. The non-pressurized fluid or gel generally differs from coatings or other structures or layers that are directly bonded to another structure (e.g., sheath 14 and/or membrane 16). Additionally, the non-pressurized fluid or gel differs from the pressurized fluid used in some rolling membrane systems in that it is not provided under pressure. Furthermore, the non-pressurized fluid may be discretely placed at the desired location rather than ubiquitously disposed throughout a lumen of system 10. This helps to reduce the amount of the lubricious fluid needed to provide the desired effect as well as minimize the exposure of the vasculature to the fluid should the fluid escape system 10. In some embodiments, the non-pressurized fluid may be provided (e.g., separately in an appropriate vessel) with system 10 so that a clinician can dispose it between sheath 14 and membrane 16 as part of the preparation of system 10 for use. Alternatively, the non-pressurized fluid may be disposed between sheath 14 and membrane 16 during the manufacture of system 10 so that it is ready to use when system 10 reaches the clinician.

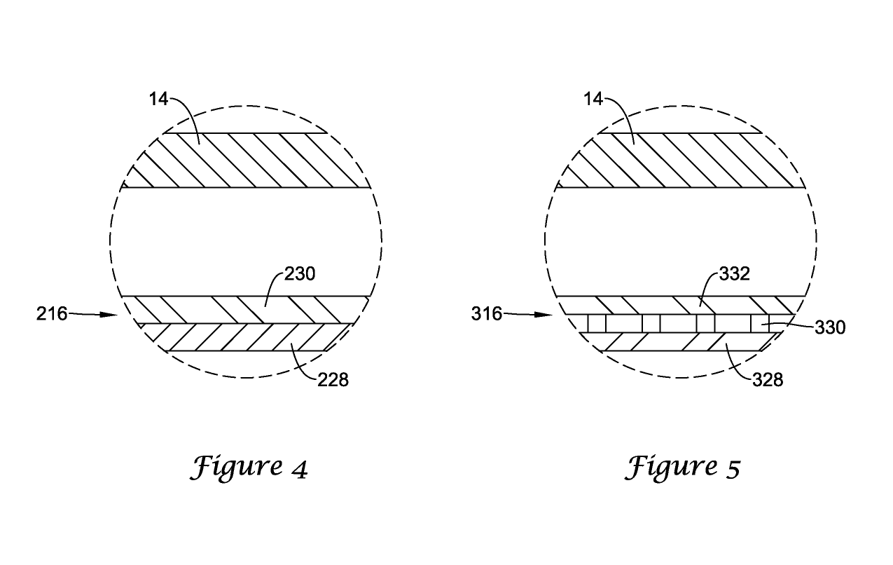

In the embodiments mentioned above, lubricious component 26/126 may comprise a distinct member that is disposed between sheath 14 and membrane 16. However, alternative embodiments are contemplated where lubricious component 26 is adhered to membrane 16 or where lubricious component 26 is a layer of or coating on membrane 16. FIG. 4 illustrates a portion of sheath 14 and a portion of an alternative membrane 216 that includes a plurality of layers. In this example, membrane 216 may include a first layer 228 and a second layer 230. Layer 228, layer 230, or both may include a lubricious material and, thus, include a lubricous component.

Multi-layer membrane 216 may have essentially the same thickness as typical membranes (e.g., about 0.0005 to about 0.007 inches or so, or about 0.001 to about 0.005 inches or so). Alternatively, membrane 216 may be slightly thicker (e.g., about 0.0007 to about 0.01 inches or so, or about 0.0015 to about 0.006 inches or so) or thinner (e.g., about 0.0004 to about 0.005 inches or so, or about 0.0007 to about 0.004 inches or so) than typical membranes. It can be appreciated that essentially membrane 216 may have any suitable thickness without departing from the spirit of the invention.

The individual layers 228/230 (and/or other layers that may be included with membrane 216) may have a thickness on the order of about 0.00003 to about 0.0004 inches or so (e.g., about 1-10 microns or so), or about 0.00003 to about 0.0001 inches or so (e.g., about 1-5 microns or so), or about 0.0001 to about 0.0004 inches or so (e.g., about 5-10 microns or so).

First layer 228 may include a suitable polymer such as a polyether block amide (e.g., PEBAX®) or any other suitable material. Second layer 230 may include a low friction or lubricious material such as polytetrafluoroethylene. Alternatively, second layer 230 may include other lubricious materials such as high-density polyethylene (HDPE), ultra high modulus polyethylene (UHMPE), or any other suitable material. Layers 228/230 may be arranged so that second layer 230 (e.g., formed of or otherwise including a lubricious material) is disposed adjacent sheath 14 so that second layer 230 can reduce friction between sheath 14 and membrane 216.

Numerous other materials are contemplated for layers 228/230 including those listed herein and other suitable materials. For example, first layer 228 may include a material having a first durometer or hardness and second layer 230 may include a material having a second durometer or hardness different from the first durometer. In at least some of these embodiments, first layer 228 may include a polyether block amide such as PEBAX® 72D and second layer 228 may include a polyether block amide such as PEBAX® 40D. One or more additional layers may also be disposed or coated onto layers 228/230.

The multi-layered membrane 216 may be formed in essentially any suitable manner. For example, membrane 216 may be coextruded. Alternatively, layers 228/230 may be provided separately and attached together, for example, using a heat bond, reflow, an adhesive, or any other suitable bonding technique. One example adhesive or tie layer that may be used to bond layers 228/230 may be OREVAC® resin, available from Arkema. In some embodiments, layers 228 may be blown, stretched, or otherwise expanded so as to thin layer 228, layer 230, or both. In other embodiments, one or more of layers 228/230 may be formed by a coating process that may including mixing the material used to form the layer with an appropriate solvent, coating the material onto a surface (e.g., on the other layer), and then allowing the solvent to volatize leaving behind the coating that forms the layer.

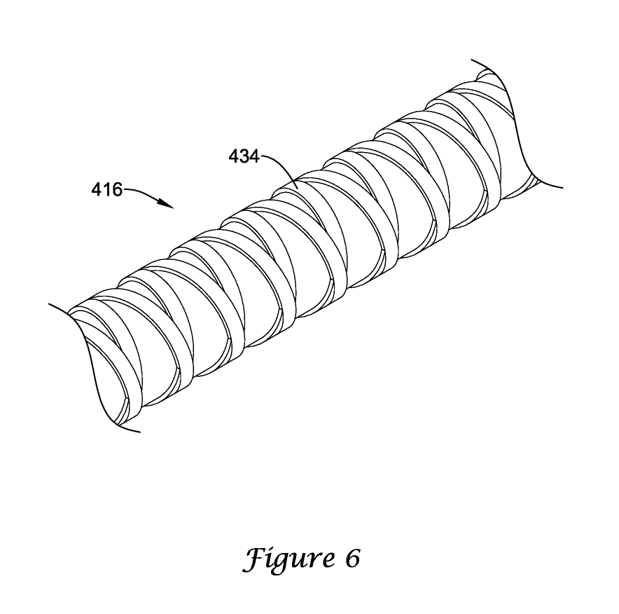

While membrane 216 is illustrated as having two layers 228/230, this is not intended to be limiting. FIG. 5 illustrates membrane 316, which may otherwise be similar in form and function to membrane 216, having three layers 328/330/332. In some embodiments, layer 332 may be formed of a lubricious material and may be disposed adjacent sheath 14 so that it can reduce friction between sheath 14 and membrane 316. The remaining layers 328/330 may include essentially any suitable material such as any of those listed herein. Alternatively, layer 328 and/or layer 330 may include a lubricious material. It can be appreciated that numerous membranes are contemplated that include additional layers including a total of four, five, six, or more layers arranged in any suitable manner.

In some embodiments, one or more of layers 328/330/332 (and/or one or more of layers 228/230) may be formed of or otherwise include a radiopaque material. For example, layer 330 may include a radiopaque material. Radiopaque materials are understood to be materials capable of producing a relatively bright image on a fluoroscopy screen or another imaging technique during a medical procedure. This relatively bright image aids the user of system 10 in determining its location. Some examples of radiopaque materials can include, but are not limited to, gold, platinum, palladium, tantalum, tungsten alloy, polymer material loaded with a radiopaque filler such as bismuth subcarbonate (Bi₂O₂(CO₃)), barium sulfate (BaSO₄), etc., and the like. In some embodiments, the radiopaque material may include any of those materials disclosed in U.S. Pat. No. 6,599,448, the entire disclosure of which is herein incorporated by reference. Additionally, other radiopaque marker bands and/or coils, or radiopaque coatings or layers may also be incorporated into the design of system 10 (e.g., at membrane 16/116/216), for example as discussed below, to achieve the same result.

The thickness of the layer including the radiopaque material (e.g., layer 328, 330, and/or 332) may vary. For example, the thickness of the layer including the radiopaque material may be about 0.0001 to about 0.001 inches or so (e.g., about 5-25 microns) or about 0.001 to about 0.004 inches or so (e.g., about 25-100 microns).

In some embodiments, the layer including a radiopaque material (e.g., layer 328, 330, and/or 332) may extend along substantially the length of membrane 316. In other embodiments, the layer including a radiopaque material may extend along a portion of the length of membrane 316. In still other embodiments, the layer including a radiopaque material may extend along multiple portions of the length of membrane 316 and, thus, form a plurality of discrete radiopaque sections.

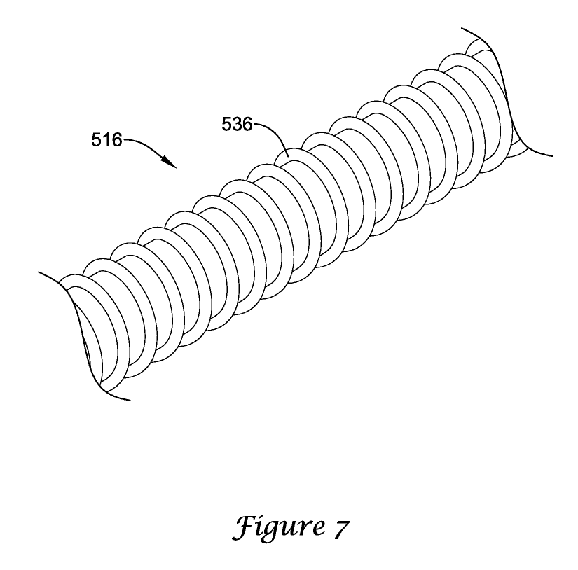

Another feature that may be desirable in a rolling membrane stent delivery system may be to increase the column strength or “pushability” of the membrane. This may help the membrane, for example, remain properly placed over stent 18 while advancing system 10 to the desired location within the anatomy. Additionally, this may reduce the chances that the membrane will buckle when sheath 14 is retracted. In at least some embodiments, any of the membranes disclosed herein may include a reinforcing member to increase the column strength thereof. For example, FIG. 6 illustrates a portion of an example membrane 416 that includes a braided reinforcing member 434. FIG. 7 illustrates a portion of an example membrane 516 that includes a coiled reinforcing member 536. These reinforcing members 434/536 may be embedded within, disposed about or along either the exterior or interior of, or otherwise included into any of the membranes disclosed herein. Accordingly, reinforcing members 434/536 may provide increased column strength to any of these membranes.

Reinforcing members 436/536 may be formed of any suitable material so as to provide the desired increase in column strength. For example, reinforcing members 436/536 may include a polymer such a polyether block amide, nylon, a poly paraphenylene terephthalamide (for example, KEVLAR®), a polymer having a relatively high durometer, or other materials. In some embodiments, reinforcing members 436/536 may include a metal or metal alloy such as those listed herein. Reinforcing members 436/536 may include a single filament or member (e.g., a single coil 536) or a plurality of filaments or members (e.g., a multi-filament braid 436). In embodiments that utilize multiple filaments, each of these filaments may include the same materials or some or all of the filaments may include different materials. Furthermore, some or all of the filaments may be or otherwise include one or more fibers or strands that are coupled together to form one or more of the filaments.

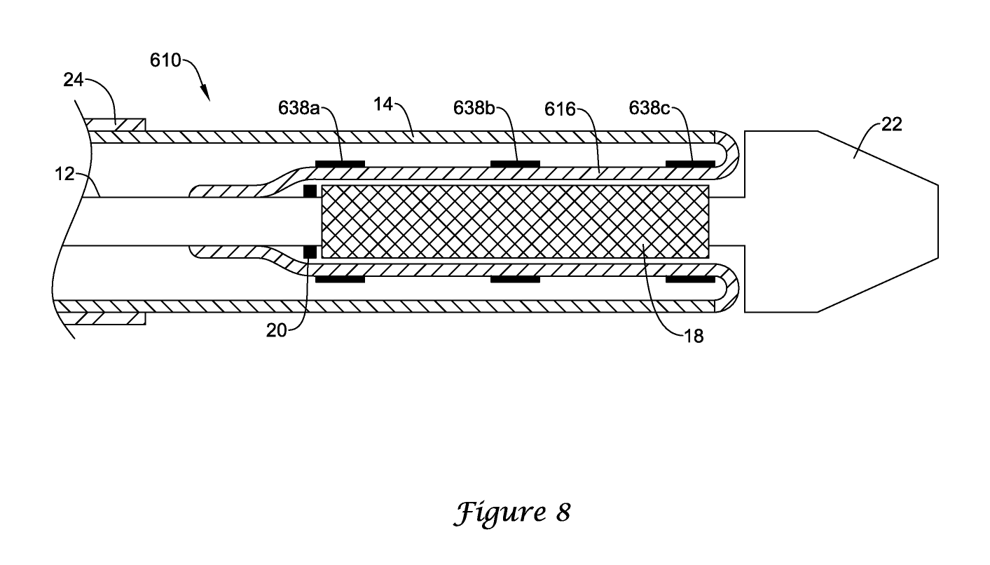

FIG. 8 illustrates another example stent delivery system 610 that may be similar in form and function to system 10. In this example, membrane 616 may have one or more radiopaque marker members or bands (bearing reference numbers 638a, 638b, and 638c) coupled thereto. For example, membrane 616 may have three radiopaque bands 638a/638b/638c as shown. In some embodiments, one or more additional bands may be added to membrane 616. For example, membrane 616 may include four, five, six, seven, eight, or more bands. In general, bands 638a/638b/638c are configured to be sufficiently flexible so that they have a minimal impact on the overall flexibility of membrane 616. For example, bands 638a/638b/638c may be sufficiently thin and/or short (e.g., in length) so that they have a minimal impact on flexibility while still providing sufficient radiopacity for visualization.

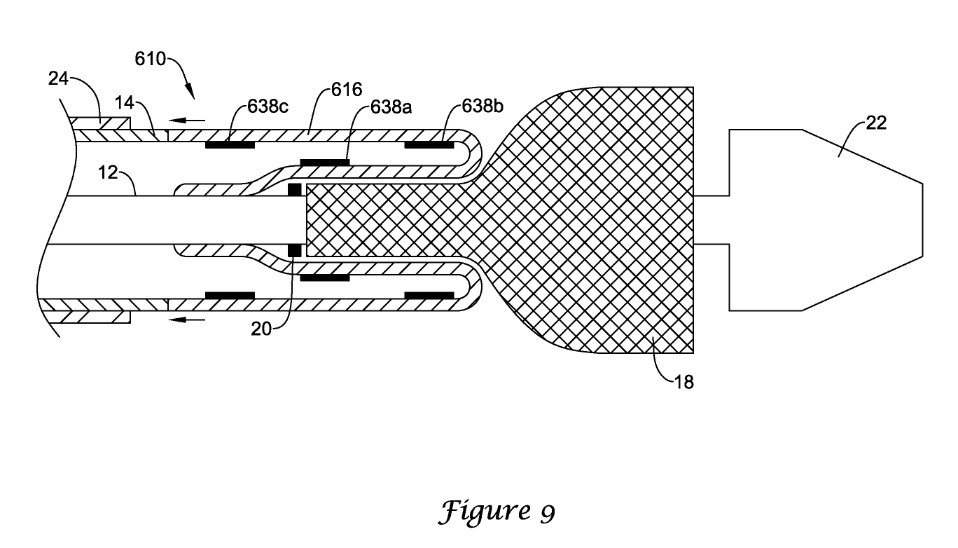

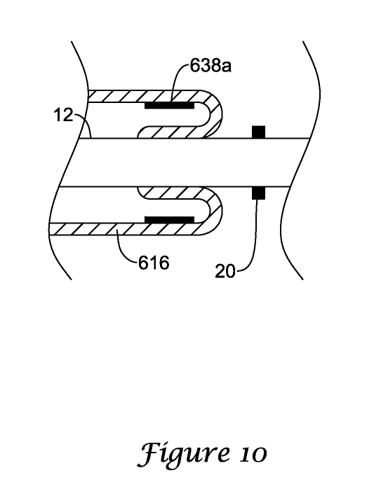

Bands 638a/638b/638c may help to aid in the visualization of system 10 and may provide a clinician with a visual indicator of the progress of the deployment of stent 18. For example, prior to deployment of stent 18 bands 638a/638b/638c may be disposed distally of bumper 20, which may also be made from or include a radiopaque material. As sheath 14 is proximally retracted, bands 638a/638b/638c may begin to migrate proximally as shown in FIG. 9. In this example, it can be seen that band 638c may be disposed proximally of bumper 20 after sheath 14 is partially refracted. This may provide a visual cue to the clinician that stent 18 is beginning to be deployed. As sheath 14 is further retracted, bands 638a/638b may also migrate proximally. Once band 638a is disposed proximally of bumper 20, for example as shown in FIG. 10, the clinician will be alerted that membrane 616 is sufficiently proximally withdrawn such that stent 18 is fully deployed.

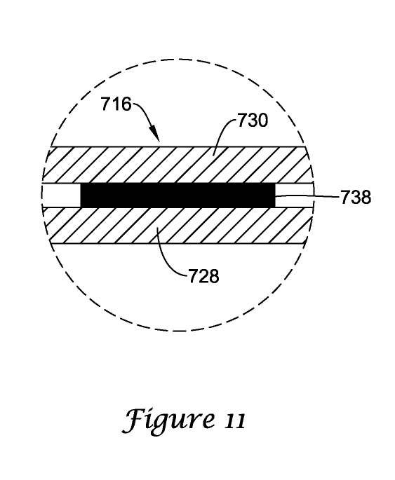

While FIGS. 8-10 illustrate membrane 616 with bands 638a/638b/638c disposed thereon, other forms of a radiopaque membrane are also contemplated. For example, radiopaque membranes are contemplated that include a layer of radiopaque material and/or a radiopaque coating as disclosed herein. FIG. 11 illustrates a portion of alternative membrane 716. Membrane 716 may include first layer 728, second layer 730, and radiopaque member 738 disposed or “sandwiched” therebetween. Member 738 may take the form of a band of radiopaque material. Alternatively, member 738 may be layer or patch of radiopaque powder (e.g., tungsten, etc.) sandwiched between layer 728/730. These embodiments may allow for sufficient visualization of membrane 716 as well as provide membrane 716 with a substantially smooth or even surface. In some embodiments, one or more layers 728/730 may include a lubricious material so as to reduce friction between sheath 14 and membrane 716.

The materials that can be used for the various components of system 10 (and/or other systems disclosed herein) may include those commonly associated with medical devices. For simplicity purposes, the following discussion makes reference to inner member 12, sheath 14, and membrane 16. However, this is not intended to limit the invention as the discussion may be applied to other similar members and/or components of members or systems disclosed herein.

Inner member 12, sheath 14, and membrane 16, and/or other components of system 10 may be made from a metal, metal alloy, polymer (some examples of which are disclosed below), a metal-polymer composite, combinations thereof, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to above, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2-0.44% strain before plastically deforming.

In some embodiments, the linear elastic and/or non-super-elastic nickel-titanium alloy is an alloy that does not show any martensite/austenite phase changes that are detectable by DSC and DMTA analysis over a large temperature range. For example, in some embodiments, there may be no martensite/austenite phase changes detectable by DSC and DMTA analysis in the range of about −60° C. to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties and has essentially no yield point.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of inner member 12, sheath 14, and membrane 16 may also be doped with, made of, or otherwise include a radiopaque material including those listed herein or other suitable radiopaque materials.

In some embodiments, a degree of MRI compatibility is imparted into system 10. For example, to enhance compatibility with Magnetic Resonance Imaging (MRI) machines, it may be desirable to make inner member 12, sheath 14, and membrane 16, in a manner that would impart a degree of MRI compatibility. For example, inner member 12, sheath 14, and membrane 16, or portions thereof, may be made of a material that does not substantially distort the image and create substantial artifacts (artifacts are gaps in the image). Certain ferromagnetic materials, for example, may not be suitable because they may create artifacts in an MRI image Inner member 12, sheath 14, and membrane 16, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers that may be used to form inner member 12, sheath 14, and membrane 16, and/or other components of system 10 may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6% LCP.

In some embodiments, the exterior surface of the system 10 may include a coating, for example a lubricious, a hydrophilic, a protective, or other type of coating. Hydrophobic coatings such as fluoropolymers provide a dry lubricity which improves device handling and exchanges. Lubricious coatings improve steerability and improve lesion crossing capability. Suitable lubricious polymers may include silicone and the like, polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, the entire disclosures of which are incorporated herein by reference.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A self-expanding stent delivery system, comprising:
an inner member;
an outer sheath slidably disposed about the inner member;
a self-expanding stent disposed between the inner member and the outer sheath;
a rolling membrane attached to the outer sheath and to the inner member; and
a lubricious component disposed adjacent the outer sheath for reducing friction between the outer sheath and the membrane.

2. The self-expanding stent delivery system of claim 1, wherein the stent delivery system is substantially free of a pressurized fluid between the outer sheath and the membrane.

3. The self-expanding stent delivery system of claim 1, wherein the lubricious component includes a floating lubricious tube.

4. The self-expanding stent delivery system of claim 1, wherein the lubricous component includes a non-pressurized lubricious fluid or gel.

5. The self-expanding stent delivery system of claim 1, wherein the rolling membrane includes a reinforcing member.

6. The self-expanding stent delivery system of claim 1, wherein the rolling membrane includes a radiopaque marker.

7. A self-expanding stent delivery system, comprising:
an inner member;
an outer sheath slidably disposed about the inner member;
a self-expanding stent disposed between the inner member and the outer sheath;
a rolling membrane attached to the outer sheath and to the inner member; and
wherein the rolling membrane includes a reinforcing member that is configured to provide the rolling membrane with increased column strength such that the reinforcing member reduces buckling of the rolling membrane when the rolling membrane is rolled back upon itself.

8. The self-expanding stent delivery system of claim 7, wherein the reinforcing member includes a braid.

9. The self-expanding stent delivery system of claim 7, wherein the reinforcing member includes a coil.

10. The self-expanding stent delivery system of claim 1, wherein the membrane includes a radiopaque marker.

11. A self-expanding stent delivery system, comprising:
an inner member;
a sheath slidably disposed about the inner member;
a self-expanding stent disposed between the inner member and the sheath;
a rolling membrane attached to the sheath and to the inner member, wherein the rolling membrane includes a radiopaque marker.

12. The stent delivery system of claim 11, wherein the radiopaque marker includes one or more radiopaque bands disposed on the membrane.

13. The stent delivery system of claim 11, wherein the membrane includes a plurality of layers and wherein the radiopaque marker includes a radiopaque layer of the membrane.

14. The stent delivery system of claim 13, wherein the membrane includes an inner layer and an outer layer, and wherein the radiopaque layer is disposed between the inner layer and the outer layer.

15. The stent delivery system of claim 11, wherein the radiopaque marker includes a radiopaque coating disposed on the membrane.

16. The stent delivery system of claim 11, further comprising a bumper disposed on the inner member.

17. The stent delivery system of claim 16, wherein the bumper includes a radiopaque material.

18. The stent delivery system of claim 17, wherein the membrane includes a radiopaque portion that includes the radiopaque member, and wherein the membrane is configured to shift between a first position where the radiopaque portion is disposed distally of the bumper and a second position where the radiopaque portion is disposed proximally of the bumper.

19. The stent delivery system of claim 11, further comprising an outer tube disposed about the sheath.

20. The stent delivery system of claim 11, wherein the rolling membrane includes a reinforcing member.
